(19) United States
(12) Patent Application Publication
(10) Pub. No.: US 2025/0121126 A1
(43) Pub. Date: Apr. 17, 2025

(54) ENDOLUMINAL TREATMENT SYSTEMS, DEVICES, AND RELATED METHODS

(71) Applicant: Boston Scientific Scimed, Inc., Maple Grove, MN (US)

(72) Inventors: Rahul PRABHU, Brighton, MA (US); Jonathan ROOT, Townsend, MA (US)

(73) Assignee: Boston Scientific Scimed, Inc., Maple Grove, MN (US)

(21) Appl. No.: 18/910,418

(22) Filed: Oct. 9, 2024

Related U.S. Application Data
(60) Provisional application No. 63/589,357, filed on Oct. 11, 2023.

(51) Int. Cl. A61M 1/00; A61M 25/09

(52) U.S. Cl. CPC A61M 1/90; A61M 25/09

(57) ABSTRACT

Medical systems, devices, and related methods are described including a medical system including a guidewire including a distal portion and a proximal portion. The distal portion may be configured to transition from an expanded configuration to a straightened configuration. In the expanded configuration, the distal portion may be configured to anchor to a target site in an anatomy. The medical system may further include a porous body coupled to a distal end of a tube. The tube may include a first lumen and a second lumen. The first lumen may be configured to receive at least the proximal portion of the guidewire and the second lumen may be configured to be coupled to a source of suction.

### CROSS-REFERENCE TO RELATED APPLICATIONS

This application claims the benefit of priority to U.S. Provisional Application No. 63/589,357, filed on Oct. 11, 2023, which is incorporated by reference herein in its entirety.

### TECHNICAL FIELD

Various aspects of this disclosure relate generally to minimally invasive medical systems, devices, and methods. In particular, aspects of the disclosure relate to medical systems, devices, and methods for endoscopic medical procedures, such as closing a wound or otherwise treating tissue.

### BACKGROUND

Endoscopic and open surgical procedures of the gastrointestinal (GI) tract include, for example, colonic resection, bariatric surgery, esophagectomy, gastric bypass, and sleeve gastrectomy, among others. These procedures may result in perforation, post-surgical anastomotic leaks, or other wounds of the GI tract. Patients with perforations, post-surgical anastomotic leaks, and/or other wounds in the GI tract have high mortality rates with limited treatment options. Options include endoscopic placement of clips or stents, endoscopic sutures or sealants, or surgical re-operation. Surgery is relatively invasive and has high morbidity and mortality rates. While endoscopic stent placement is a less invasive option, the stent can migrate from the intended location and/or wall off infection at a treatment site, inhibiting drainage.

The medical systems, devices, and methods of the current disclosure may rectify some of the deficiencies described above or address other aspects of the art.

### SUMMARY

Each of the aspects disclosed herein may include one or more aspects of the features described in connection with any of the other disclosed aspects.

According to some aspects of the present disclosure, a medical system may include a guidewire including a distal portion and a proximal portion. The distal portion may be configured to transition from an expanded configuration to a straightened configuration. In the expanded configuration, the distal portion may be configured to anchor to a target site in an anatomy. The medical system may include a porous body coupled to a distal end of a tube. The tube may include a first lumen and a second lumen. The first lumen may be configured to receive at least the proximal portion of the guidewire and the second lumen may be configured to be coupled to a source of suction.

According to some aspects, in the expanded configuration, the distal portion may have a sinuous, coil, or spiral shape. In some examples, the distal portion may include a plurality of securement structures. In some examples, the plurality of securement structures may include at least one of a burr, a barb, a ridge, a protrusion, or an adhesive. In some examples, the proximal portion of the guidewire may include a portion that is straight. In some examples, the distal portion may be configured to transition from the expanded configuration to the straightened configuration when a proximal end of the distal portion moves proximally relative to a distal end of the distal portion. In some examples, the proximal portion and the distal portion may be integrally formed. In some examples, in the expanded configuration, the distal portion may be wider in a direction perpendicular to a longitudinal axis of the proximal portion than the proximal portion is. In some examples, the distal portion may biased to the expanded configuration. In some examples, the tube may include a main portion including an outer wall and an inner wall. The inner wall may separate the first lumen and the second lumen. In some examples, a proximal end of the tube may include a first branch and a second branch extending proximally from the main portion. The first lumen may extend through the first branch and the second lumen may extend through the second branch. In some examples, the outer wall may be a first outer wall. The first branch may include a second outer wall and the second branch may include a third outer wall. The second outer wall of the first branch of the tube and the third outer wall of the second branch of the tube may each be continuous with the first outer wall of the main portion of the tube. In some examples, the second branch may be configured to couple to the source of suction to deliver negative pressure to the porous body via the second lumen of the tube. In some examples, the medical system may further include a connector configured to be removably coupled to a proximal end of the main portion. The connector may include a first tube defining a third lumen and a second tube defining a fourth lumen. In a state in which the connector may be coupled to the proximal end of the main portion, the third lumen may be in fluid communication with the first lumen and the fourth lumen may be in fluid communication with the second lumen. In some examples, a proximal end of the proximal portion of the guidewire may be configured to be inserted into the porous body and into a distal opening of the first lumen.

According to some aspects of the present disclosure, a medical system may include a guidewire including a distal portion and a proximal portion. The distal portion may be biased to an expanded configuration. The medical system may include a porous body coupled to a distal end of a tube. The tube may include a first lumen and a second lumen. A proximal end of the proximal portion may be configured to be inserted into a distal opening of the first lumen so that the porous body and the tube may be advanced along and relative to the guidewire in order to position the porous body and the tube at a target site. In some examples, in the expanded configuration, the distal portion may have a sinuous, coil, or spiral shape.

According to some aspects of the present disclosure, a method of treating a target site of a body lumen of a patient via a medical device may include advancing a distal portion of a guidewire into the target site. The distal portion of the guidewire may be biased to an expanded configuration to anchor the distal portion relative to the target site. The method may further include inserting a proximal end of the guidewire through a porous body and into an opening of a first lumen at a distal end of a tube coupled to the porous body, moving the tube and the porous body distally along the guidewire to position the porous body proximate to the target site, removing the guidewire from the body lumen by pulling the guidewire proximally through the porous body and through the first lumen of the tube, and supplying a negative pressure to the porous body through a second lumen of the tube. In some examples, removing the guidewire from the body lumen may include pushing the porous body distally while pulling the guidewire proximally. In some examples, pulling the guidewire proximally may transition the guidewire from the expanded configuration to a straightened configuration.

### BRIEF DESCRIPTION OF THE DRAWINGS

The accompanying drawings, which are incorporated in and constitute a part of this specification, illustrate various aspects of this disclosure and together with the description, serve to explain the principles of the disclosure.

FIGS. 1A-1E depict an exemplary medical system, in accordance with some aspects of the present disclosure.

FIGS. 2A-2D depict an exemplary method of using the exemplary medical system of FIGS. 1A-1E, in accordance with some aspects of the present disclosure.

FIGS. 3A and 3B depict another exemplary medical system, in accordance with some aspects of the present disclosure.

FIG. 4 depicts an exemplary medical device, in accordance with some aspects of the present disclosure.

FIG. 5 depicts an exemplary medical device, in accordance with some aspects of the present disclosure.

### DETAILED DESCRIPTION

Particular aspects of the present disclosure are described in greater detail below. The terms and definitions provided herein control, if in conflict with terms and/or definitions incorporated by reference.

The terms “proximal” and “distal” are used herein to refer to the relative positions of the components of exemplary medical devices. As used herein, “proximal” refers to a position relatively closer to the exterior of the body or closer to an operator using the medical device. In contrast, “distal” refers to a position relatively further away from the operator using the medical device, or closer to the interior of the body.

As used herein, the terms “comprises,” “comprising,” “including,” “includes,” “having,” “has” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.”

Further, relative terms such as, for example, “about,” “substantially,” “approximately,” etc., are used to indicate a possible variation of ±10% in a stated numeric value or range.

Endoluminal vacuum therapy (EVT or EVAC, and referred to herein as EVAC) is a procedure to treat wounds, such as post-surgical leaks or perforations in the gastrointestinal tract (GI) following a surgical or endoscopic procedure, such as colonic resection, bariatric surgery, or esophagectomy. In EVAC, negative pressure is delivered to the wound site in the GI tract, for example, through a nasogastric tube having a sponge-like material or foam (e.g., vacuum sealed foam) sutured at its distal end. A proximal end of the tube may be connected to a collection container. The foam is placed endoscopically into the perforation, leak, or other wound. In some examples, EVAC includes endoluminal placement of a foam or other like material into the wound (e.g., target) site, including a perforation, a leak, a cyst, an anastomosis, etc. Placement of the material may be via a catheter, scope (endoscope, bronchoscope, colonoscope, duodenoscope, gastroscope, etc.), tube, or sheath, inserted into the GI tract via a natural orifice. The orifice can be, for example, the nose, mouth, or anus, and the placement can be in any portion of the GI tract, including the esophagus, stomach, duodenum, large intestine, or small intestine. Placement of the material can also be in other organs reachable via the GI tract (e.g., the colon). Negative pressure then is applied.

The foam in the wound, along with the negative pressure, may accelerate

healing by encouraging local tissue granulation at a wound site. The foam may be replaced with increasing smaller sizes of foam as the wound heals and closes. Devices and systems, including delivery systems, suited for EVAC are limited. During EVAC, rat-tooth forceps or another accessory device may be extended through a working channel of a scope and used to guide a foam to a wound site as the scope is navigated to the wound site, which may be difficult to accomplish in narrow body lumens (e.g., the esophagus). For example, use of both a scope and rat-tooth forceps may overcrowd a narrow body lumen. Furthermore, at least two operators may be needed to guide the scope and the rat-tooth forceps, along with the foam, to the wound site simultaneously. In addition, standard guidewires may not be suitable for delivering a foam to a target site of a body lumen during EVAC. For example, a standard guidewire may migrate from a target site within a body lumen as a scope is removed from the body lumen and/or as a foam is placed into the target site.

Aspects of this disclosure include systems, devices, and methods to improve delivery of a foam to a target site and/or to reduce the number of operators needed to perform an EVAC procedure. Components of the systems and/or devices described herein may be packaged as a kit for EVAC. For example, the systems disclosed herein may include a guidewire having a coil, sinuous, helical, spiral, winding, or other non-straight shape at a distal portion of the guidewire in a relaxed, natural configuration of the guidewire to facilitate anchoring of the distal portion of the guidewire within a target site and prevent migration of the distal portion of the guidewire from the target site. The systems disclosed herein may further include a porous body coupled to a distal end of a tube including two independent lumens. In some examples, one lumen may be configured to receive the guidewire to navigate the porous body and the tube to a target site and the other lumen may be configured to supply a negative pressure to the porous body.

Reference will now be made in detail to examples of the present disclosure described above and illustrated in the accompanying drawings. Wherever possible, the same reference numbers will be used throughout the drawings to refer to the same or like parts.

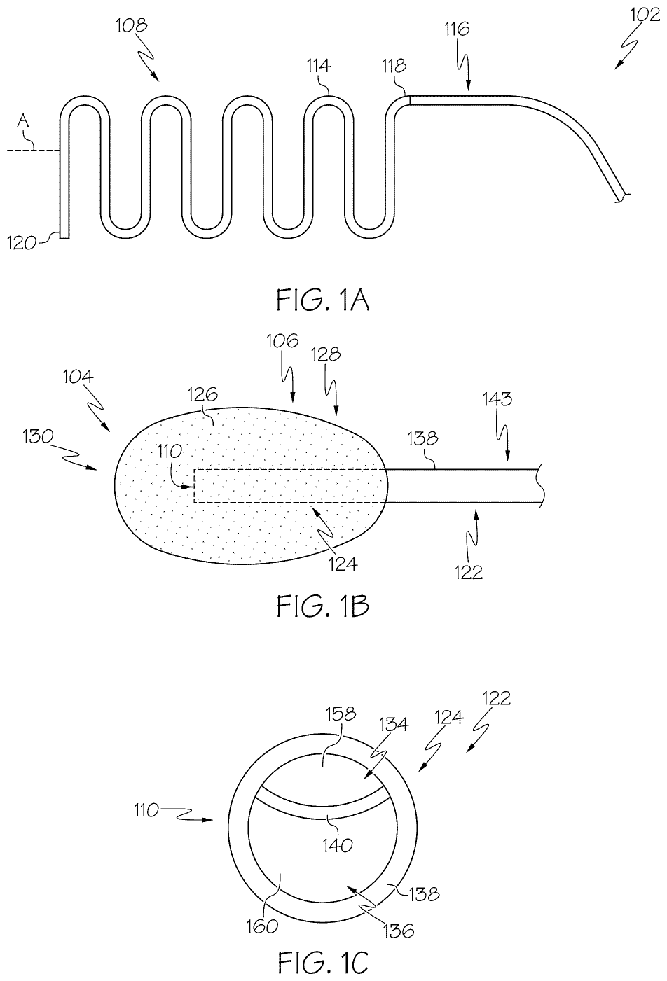

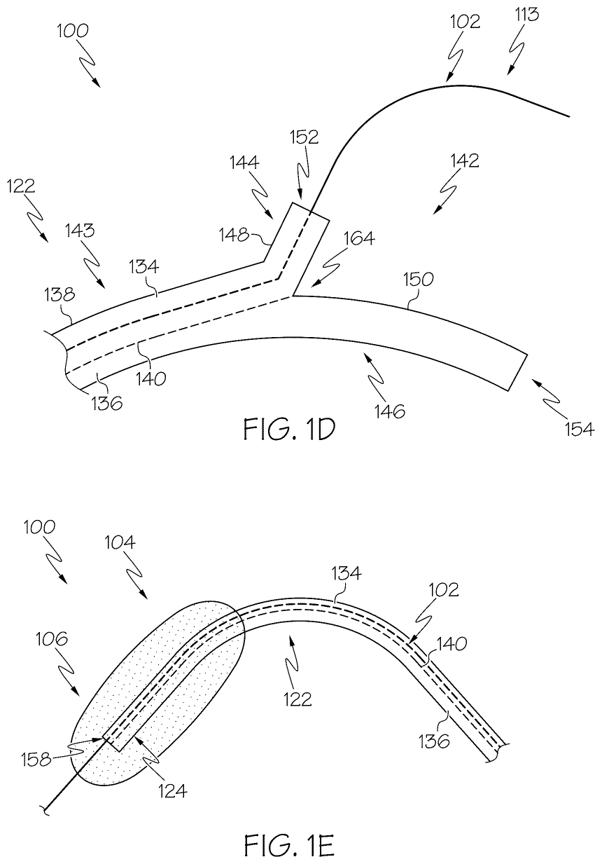

FIGS. 1A-1E illustrate an exemplary medical system 100, including a guidewire 102 (FIGS. 1A, 1D, and 1E) and an EVAC device 104 (FIGS. 1B-1E), that may be inserted into a patient to help the healing of a wound, an anastomosis, or the like, or to otherwise treat tissue, as described above. FIG. 1A illustrates a distal end portion of guidewire 102 in a relaxed, natural configuration. Unless otherwise specified herein, guidewire 102 may have any features of any guidewire known in the art. For example, guidewire 102 may have a length and/or width that is sized to be received within a working channel of an introduction device (e.g., an endoscope or other type of scope device) and within a lumen of EVAC device 104, as discussed below. Guidewire 102 may be sufficiently flexible so as to be extended through the working channel of the introduction device. In some examples, guidewire 102 may include a coating that promotes smooth tracking and enhances tactile sensation.

Guidewire 102 may include a distal portion 108 and a proximal portion 116. Proximal portion 116 may extend proximally from a proximal, first end 118 of distal portion 108 to a proximalmost end 113 of guidewire 102 (shown in FIG. 1D). Distal portion 108 of guidewire 102 may be configured to transition between an expanded configuration (shown in FIG. 1A) and a straightened configuration (a first strained configuration), in which distal portion 108 may be straightened, as described below. Distal portion 108 may be biased towards the expanded configuration in the relaxed, natural configuration of guidewire 102.

In the expanded (relaxed, biased) configuration, distal portion 108 of guidewire 102 may preferably have a coil shape. However, it will be appreciated that distal portion 108 of guidewire 102 may have any suitable shape, such as a sinuous shape, a spiral, a helix, or any non-straight shape that may include bends, curves, windings, and/or rings. As used herein, the term “straight” as pertaining to guidewire 102 includes a guidewire 102 that may have passive or active curves due to flexibility or steering but which may have a straight shape in a natural, relaxed configuration (e.g., when laid on a table). The term “straight” contrasts to a shape of distal portion 108, which may have preformed bends, curves, windings, rings, etc. which may be formed by, for example, heat treating distal portion 108 around a mandrel or other shaping structure. For example, guidewire 102 may comprise Nitinol (nickel titanium).

In some examples, distal portion 108 may be straightened from the expanded, relaxed, natural configuration to adopt the straightened configuration (the first strained configuration). Distal portion 108 may include proximal, first end 118 and a distal (e.g., distalmost), second end 120 (which may be a tip of distal portion 108). Second end 120 may be a free end—that is, an end of distal portion 108 that is not connected to another structure. Distal portion 108 may be straightened by, for example, pulling second end 120 of distal portion 108 distally relative to first end 118. Additionally or alternatively, distal portion 108 may be straightened by pulling first end 118 of distal portion 108 proximally relative to second end 120. In the straightened configuration, bends, curves, etc. of distal portion 108 may be removed or decreased, such that distal portion 108 is straighter than in the expanded configuration.

In some examples, distal portion 108 of guidewire 102 may be compressed to a compressed configuration (a second strained configuration). In some examples, distal portion 108 may be compressed from the relaxed, natural configuration by, e.g., pushing or otherwise moving second end 120 proximally relative to first end 118 and/or by moving first end 118 distally relative to second end 120. In other words, second end 120 and first end 118 may be moved toward each other.

Distal portion 108 may return to the expanded configuration from the straightened configuration or the compressed configuration once force(s) are removed, due to the shape memory properties of distal portion 108, discussed above.

Portions 108 and 116 of guidewire 102 may be flexible. In some examples, distal portion 108 and proximal portion 116 may be composed of the same material (e.g., Nitinol). In other examples, distal portion 108 may be formed of a separate material from proximal portion 116. For example, proximal portion 116 may be formed from, for example, stainless steel, with or without coatings such as polytetrafluoroethylene (PTFE). Proximal portion 116 and distal portion 108 may be integrally formed from a single piece of material or may be formed from separate pieces of material and coupled together.

Distal portion 108 of guidewire 102 may be configured to be inserted within a target site (e.g., a wound cavity) within a body lumen. Guidewire 102 may be received by or inserted within EVAC device 104 to guide or navigate EVAC device 104 to a target site, which will be discussed in greater detail below.

The expanded configuration of distal portion 108 may allow distal portion 108 to anchor or otherwise remain positioned within a target site without moving or migrating from the target site unless a sufficient force is applied. As discussed in further detail above, in the expanded configuration, distal portion 108 of guidewire 102 may have a coil, a spiral, a sinuous, or any non-uniform shape that may include bends, curves, and/or rings, which may be formed, for example, by heat treating distal portion 108 around a mandrel. As shown in FIG. 1A, in the expanded configuration, distal portion 108 may have a uniform width (i.e., in a direction perpendicular to a longitudinal axis A) along a longitudinal length of distal portion 108. In the expanded configuration, distal portion 108 may be wider in a direction perpendicular to a longitudinal axis of proximal portion 116 than proximal portion 116 is. In other examples, distal portion 108 may have a non-uniform width from proximal, first end 118 to distal, second end 120. For example, a width of distal portion 108 may increase (taper outwardly) between proximal, first end 118 to distal, second end 120. Alternatively, a width of distal portion 108 may decrease (taper inwardly) between proximal, first end 118 and distal, second end 120. In further alternatives, a longitudinally middle portion of distal portion 108 may bulge outwardly as compared to proximal and distal portions of distal portion 108. Distal portion 108 may include any suitable number of bends, curves (e.g., curves 114 illustrated in FIG. 1A), and/or rings/windings. In some examples, a size of distal portion 108 may be selected based on a location or size of a target site. For example, guidewire 102 may be provided in a kit having multiple guidewires 102 with different sizes of distal portions 108.

FIGS. 1B and 1C illustrate a distal portion of exemplary EVAC device 104, including a foam 106, or other porous body, and a tube 122 or other type of conduit. Foam 106 may have any features of any foam that is known in the art for use in EVAC procedures. For example, foam 106 may include an open-cell foam. Foam 106 may include openings 126 on an outer surface and/or interior thereof. Openings 126 may be any hole, pore, or channel. Openings 126 may include interconnecting channels and/or pores throughout foam 106. For example, openings 126 may be pores of foam 106. Openings 126 may have different sizes and/or shapes. Features of openings 126 (e.g., a size and shape of pores of foam 106) may be selected based on a location of treatment within the body, properties of a wound to be treated, a stage of treatment, or other factors. Foam 106 is illustrated as having an ovoid shape, but may be any shape, including spherical, cylindrical, prismatic, cuboidal, irregular, or the like. Foam 106 may be compressed into a lower profile during insertion to a target site and may expand during deployment at a target site.

Foam 106 may include any suitable biocompatible material that may absorb liquids and/or permit liquid or other materials to pass therethrough via, e.g., negative pressure applied to foam 106. The material of foam 106 may be flexible, compressible, porous, hydrophilic, sterile, and/or disposable. Suitable materials include polyurethanes, esters, ethers, composite materials, and/or other medical-grade materials.

Foam 106 may be fixedly or releasably attached to a distal end 124 of tube 122. For example, distal end 124 of tube 122 may be attached to foam 106 via sutures or other ties, an adhesive, a shrink-wrapped material, elastic(s), or the like. In one example, a recess (not shown) may be provided in foam 106 (e.g., a proximal end 128 of foam 106) to receive distal end 124 of tube 122. Although tube 122 is illustrated as terminating proximal to a distal end 130 of foam 106 in FIG. 1B, it will be appreciated that tube 122 may extend fully through foam 106 such that a distal face 110 at distal end 124 of tube 122, including one or more distal-facing openings (discussed below), is aligned with or adjacent to distal end 130 of foam 106.

FIG. 1C illustrates a proximally-facing plan view of distal face 110 of distal end 124 of tube 122. FIG. 1D illustrates a proximal portion of medical system 100, including a proximal end 142 of tube 122. Tube 122 may include two independent or separate open-ended lumens (i.e., lumens having proximal and distal openings). In some examples, tube 122 may be a multi-lumen extrusion. Tube 122 may include a first lumen 134 and a second lumen 136 extending from distal end 124 of tube 122 to proximal end 142 of tube 122. Each lumen 134, 136 may be defined by inner and/or outer walls of tube 122, which will be discussed in greater detail below. First lumen 134 may include a distal-facing opening 158 at distal face 110 of tube 122 and a proximal-facing opening 152 at proximal end 142 of tube 122. Second lumen 136 may include a distal-facing opening 160 at distal face 110 of tube 122 and a proximal-facing opening 154 at proximal end 142 of tube 122.

Tube 122 may include a main portion 143 defined by a first outer wall 138 extending from distal end 124 of tube 122, as shown in FIGS. 1B and 1C, to a fork 164 at proximal end 142 of tube 122, as shown in FIG. 1D. Portions of lumens 134 and 136 extending through main portion 143 may be separated or divided by an inner wall 140 extending within/across main portion 143 and along an entire length (i.e., in a longitudinal direction) of main portion 143. Inner wall 140 may have any suitable shape (e.g., curved, as shown in FIG. 1C, or straight).

Tube 122 may include a first branch or branched portion 144 and a second branch or branched portion 146 that fork off or branch off (and extend from) main portion 143 at fork 164. For example, branches 144 and 146 may extend proximally from main portion 143 at fork 164, and in opposite directions or otherwise away from each other. In some examples, first branch 144 may be angled relative to a longitudinal axis of main portion 143 and second branch 146 may extend along the longitudinal axis of main portion 143, and vice versa. First branch 144 of tube 122 may be defined by a second outer wall 148 and second branch 146 of tube 122 may be defined by a third outer wall 150, and each outer wall 148, 150 may be continuous with first outer wall 138 of main portion 143. In examples, main portion 143 and branches 144, 146 may be integrally formed with one another, from a same piece of material (e.g., a single molded piece). In alternatives, main portion 143 and branches 144, 146 may be formed from separate pieces that are coupled (e.g., fixedly coupled) to one another.

As mentioned above, distal end 124 of tube 122 may include distal-facing openings 158, 160 of lumens 134, 136, as shown in FIG. 1C, and proximal-facing openings 152, 154 of lumens 134, 136 at proximal end 142, as shown in FIG. 1D. Distal-facing openings 158, 160 of lumens 134, 136 may be defined by portions of first outer wall 138 and portions of inner wall 140. Proximal-facing opening 152 of first lumen 134 may be defined by second outer wall 148 of first branch 144 and proximal-facing opening 154 of second lumen 136 may be defined by third outer wall 150 of second branch 146. First lumen 134 may continuously extend from distal-facing opening 158, through main portion 143, through first branch 144, and to proximal-facing opening 152. Thus, first lumen 134 may be defined by portions of first outer wall 138 and portions of inner wall 140 of main portion 143, and by second outer wall 148 of first branch 144. Second lumen 136 may continuously extend from distal-facing opening 160, through main portion 143, through second branch 146, and to proximal-facing opening 154. Thus, second lumen 136 may be defined by portions of first outer wall 138 and portions of inner wall 140 of main portion 143, and by third outer wall 150 of second branch 146.

Inner wall 140 and branches 144, 146 may allow for lumens 134, 136 to be separate and independent of one another along an entire length of tube 122. First lumen 134 may be configured receive guidewire 102 and second lumen 136 may be configured to supply a negative pressure to foam 106. As shown in FIG. 1C, second lumen 136 may have a larger cross-sectional area than first lumen 134. It will be appreciated, however, that lumens 134 and 136 may be of any size and/or shape.

As shown in FIGS. 1D and 1E, guidewire 102 may be received within first lumen 134 and may be movable relative to EVAC device 104 (or EVAC device 104 may be movable relative to guidewire 102). For example, once guidewire 102 is positioned in a target site (e.g., in a cavity), as discussed in further detail below, proximalmost end 113 of guidewire 102 may be inserted through foam 106 and through distal-facing opening 158 of first lumen 134 at distal end 124 of tube 122. EVAC device 104 may be moved distally along and relative to guidewire 102 to the target site, such that proximalmost end 113 of guidewire 102 may extend out of or exit out of proximal-facing opening 152 of first lumen 134. As discussed further below, guidewire 102 may subsequently be removed from tube 122.

Proximal-facing opening 154 of second lumen 136 may be coupled to a vacuum source (not shown). The vacuum source may supply a negative pressure to foam 106 via distal-facing opening 160 of second lumen 136 (and/or side openings of second lumen 136). For example, a negative pressure of approximately 125 mm Hg, or approximately 2.5 pounds per square inch (PSI), may be supplied to foam 106 via distal-facing opening 160 of second lumen 136. Other suitable amounts of negative pressure may be used. The negative pressure may pull fluid, material, and/or other debris into second lumen 136 via openings 126 of foam 106, which may promote healing at a target site. Second branch 146 may extend further proximally, or in other words, may be longer than first branch 144, such that proximal-facing opening 154 of second lumen 136 may be proximal to proximal-facing opening 152 of first lumen 134, which may allow second branch 146 to be coupled to the vacuum source.

Although tube 122 is illustrated as a single tube defining first lumen 134 and second lumen 136, in an alternative, a first separate tube defining first lumen 134 may be fused or otherwise coupled together with a second separate tube defining second lumen 136. For example, portions of outer walls of the first tube and the second tube may extend adjacent one another or be fused together up to, e.g., fork 164, while proximal portions of the first tube and the second tube may extend separately and away from each other, e.g., similar to first branch 144 and second branch 146.

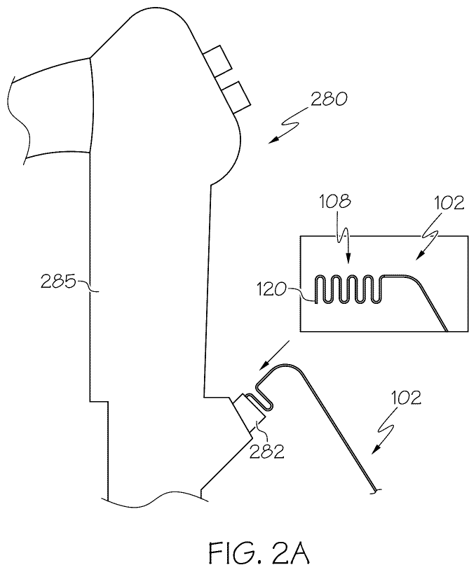

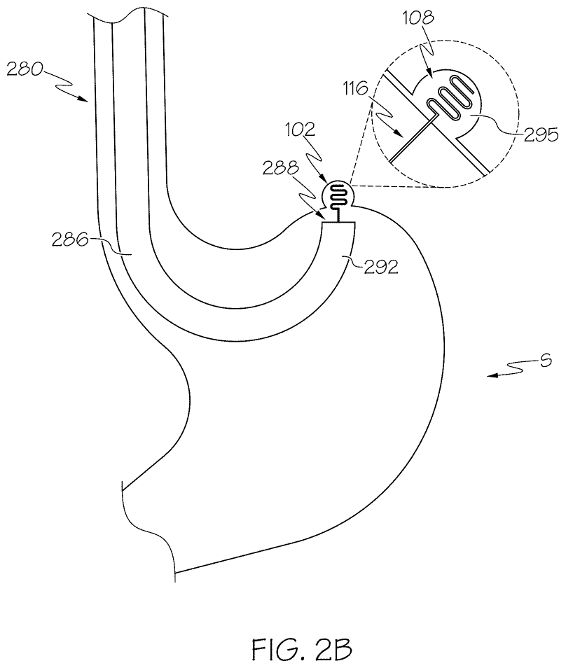

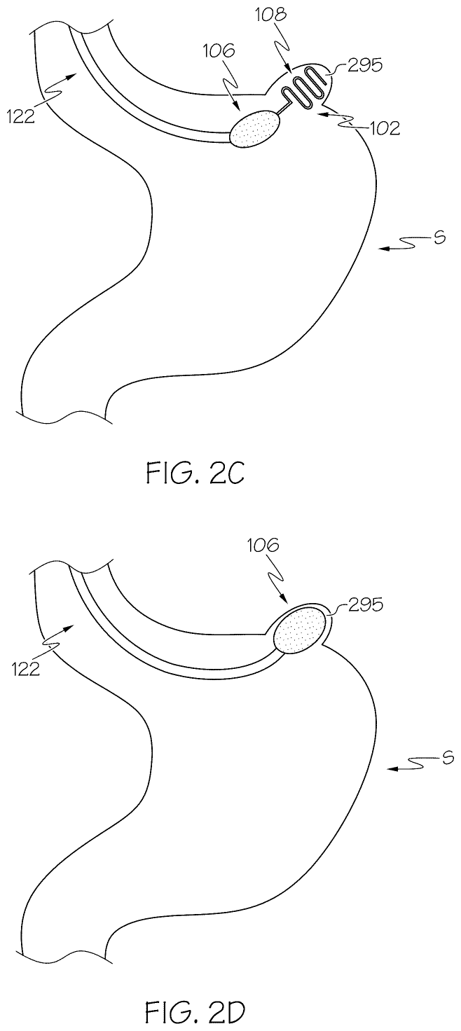

An exemplary method of using medical system 100 will now be described. FIGS. 2A-2D illustrate an exemplary method of delivering EVAC device 104 to a target site 295 (e.g., a wound, a leak, etc.) within a body lumen or anatomy of a patient (e.g., a portion of the GI tract, e.g., the stomach S) to treat target site 295 (FIGS. 2B-2D).

To position EVAC device 104 at target site 295, an operator may insert second end 120 of distal portion 108 of guidewire 102 into a port 282 on a handle 285 of an endoscope 280 (or other medical device), as shown in FIG. 2A. As mentioned above, the flexibility of distal portion 108 of guidewire 102 may allow for distal portion 108 of guidewire 102 to be easily inserted through port 282. In some examples, the operator may manually extend/straighten distal portion 108 before inserting second end 120 into port 282. Alternatively, a size of distal portion 108 may be such that it may be inserted into port 282 in an expanded configuration. Guidewire 102 may be extended through port 282 and through a working channel of endoscope 280. The working channel of endoscope 280 may extend through a shaft 286 of endoscope 280 and to a working channel opening 288 at a distal end 292 of shaft 286. Guidewire 102 may be extended distally through the working channel of endoscope 280 until distal portion 108 is positioned proximate to working channel opening 288. Proximalmost end 113 of guidewire 102 may extend outside of port 282.

The operator may then insert shaft 286 of endoscope 280 into the patient via a natural orifice (e.g., the mouth) and position distal end 292 of shaft 286 proximate to target site 295, as shown in FIG. 2B. The operator may then advance or move guidewire 102 distally out of working channel opening 288 and position and/or anchor distal portion 108 of guidewire 102 within target site 295. In alternatives, shaft 286 may be navigated to target site 295 before guidewire 102 is inserted into port 282. Endoscope 280 may then be moved proximally (retracted) relative to guidewire 102 and removed from the patient's body. Distal portion 108 may be in the expanded, biased configuration, and may thus be retained at target site 295 (e.g., within a defect). Endoscope 280 may therefore be removed from the patient's body without moving distal portion 108 of guidewire 102 from target site 295.

Guidewire 102 may then be used to guide or navigate EVAC device 104 to target site 295. Proximalmost end 113 of guidewire 102 may be inserted into or received within distal-facing opening 158 of first lumen 134 of tube 122. Foam 106 and tube 122 may then be moved/fed distally along proximal portion 116 of guidewire 102 and navigated to target site 295. As shown in FIG. 2C, foam 106 may be positioned proximate to (e.g., adjacent to) distal portion 108 of guidewire 102 within target site 295. As foam 106 and tube 122 are moved distally along proximal portion 116 of guidewire 102 to target site 295, branch 144 may remain distal to proximalmost end 113 of guidewire 102 such that proximalmost end 113 of guidewire 102 extends beyond or out of proximal-facing opening 152 of first lumen 134, as shown in FIG. 1D.

To position foam 106 within target site 295, an operator may simultaneously advance or move tube 122 distally to push or move foam 106 into target site 295 and pull proximalmost end 113 of guidewire 102 (or another part of proximal portion 116) to retract or proximally move distal portion 108 of guidewire 102 relative to foam 106. Relative forces between foam 106 and distal portion 108 (e.g., pushing or compressing foam 106 against distal portion 108) may provide a sufficient compressive force such that proximal movement of guidewire 102 may cause distal portion 108 to move proximally into foam 106, thereby transitioning distal portion 108 to the straightened configuration and allowing it to be retracted through foam 106 and into first lumen 134 of tube 122. It will be appreciated that more proximal portions of distal portion 108 (e.g., portions near first end 118) proximal end may transition to the straightened configuration prior to more distal portions of distal portion 108 (e.g., portions near second end 120), because the more proximal portions of distal portion 108 may be pulled into foam 106/tube 122 before more distal portions of distal portion 108.

With distal portion 108 of guidewire 102 positioned within first lumen 134 of tube 122, guidewire 102 may be removed from the patient's body by retracting or pulling guidewire 102 proximally through first lumen 134 of tube 122 and out of proximal-facing opening 152 of first lumen 134. Foam 106 and tube 122 may remain in the patient's body, as shown in FIG. 2D. The operator may then connect a vacuum source (not shown) to second branch 146 of tube 122 to supply negative pressure to foam 106 through second lumen 136, which may pull fluid, material, and/or other debris from target site 295 into second lumen 136 to promote healing.

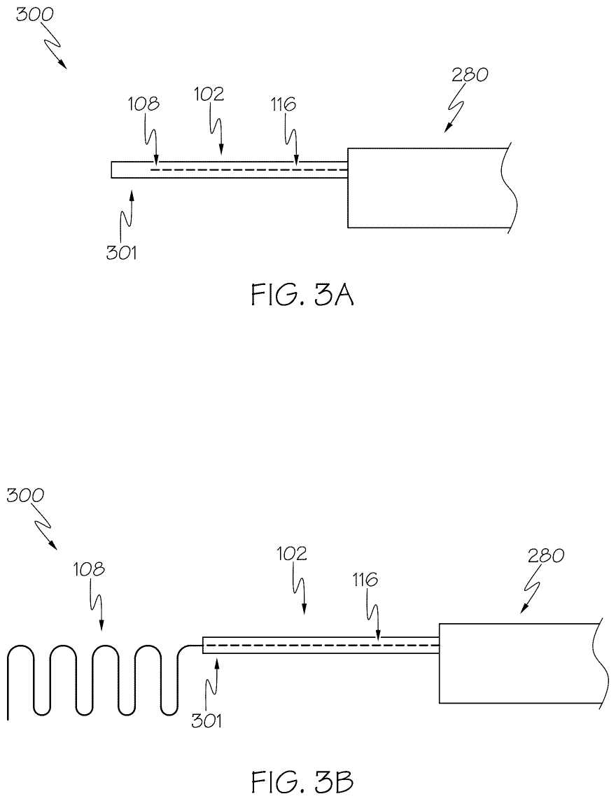

FIGS. 3A-3B depict an alternative medical system 300 having any of the features of medical system 100, described above, except as provided herein. In addition to the elements of medical system 100 described above, medical system 300 may further include an overtube 301 having open proximal and distal ends for delivering guidewire 102 to a target site (e.g., target site 295). Overtube 301 may facilitate movement of guidewire 102 through a working channel of an endoscope, such as endoscope 280 (or other medical device). Guidewire 102 may be inserted into or loaded into overtube 301 (or may come pre-loaded in overtube 301), such that distal portion 108 of guidewire 102 may be in the straightened configuration, as shown in FIG. 3A, during insertion to a target site. For example, an operator may insert overtube 301, along with guidewire 102 positioned therein, into the working channel of endoscope 280. The operator may move overtube 301, along with guidewire 102, distally through the working channel. With a distal portion of endoscope 280 positioned proximate to the target site, the operator may move overtube 301, along with guidewire 102, distally out of the working channel and position distal portion 108 of guidewire 102 within the target site. Overtube 301 may then be moved proximally (retracted) relative to guidewire 102 to transition distal portion 108 of guidewire 102 from the straightened configuration to the expanded (relaxed, natural) configuration, as shown in FIG. 3B. Overtube 301 may improve visualization during delivery and placement of guidewire 102 into a target site. Overtube 301 may also be helpful in positioning guidewire 102 in difficult target sites (e.g., a perforation at the top of the stomach after a sleeve gastrectomy).

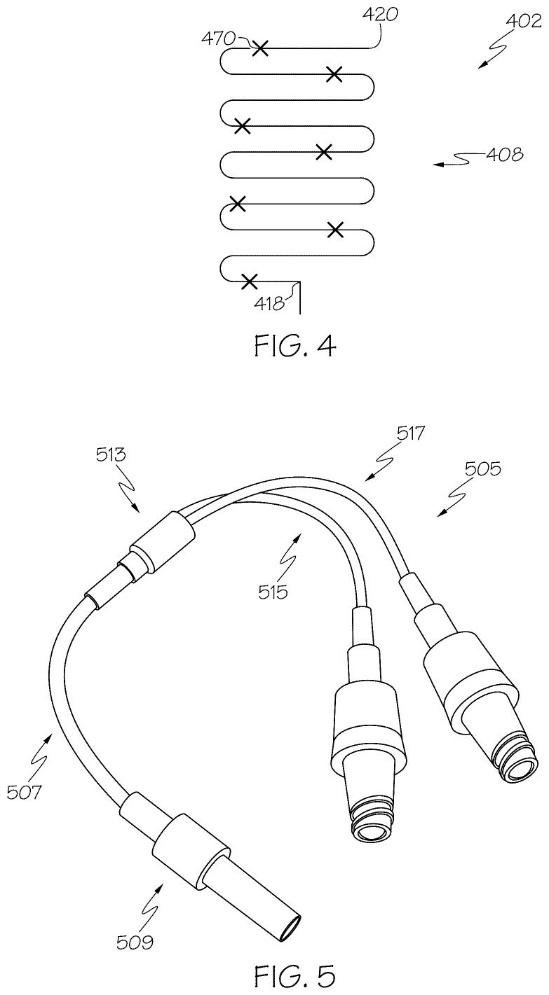

FIG. 4 illustrates an alternative guidewire 402, which may have any of the features of guidewire 102, unless otherwise specified. Guidewire 402 may be used in conjunction with any of the aspects discussed above, including overtube 301. Guidewire 402 may include one or more securement structures 470. One or more securement structures 470 may be disposed along an entire length (e.g., from a proximal, first end 418 to a distal, second end 420) of a distal portion 408 of guidewire 402. Securement structures 470 may include burrs, barbs, ridges, protrusions, adhesive, or any other structure that may further facilitate anchoring or securement of distal portion 408 of guidewire 402 within a target site. In some examples, securement structures 470 may be evenly spaced apart along distal portion 408 of guidewire 402. In alternatives, securement structures 470 may be randomly spaced apart along distal portion 408 of guidewire 402. In some examples, only portions (e.g., a proximal portion or a distal portion) of distal portion 408 may include securement structures 470. In other examples, only one side of distal portion 408, when it is in the expanded configuration, may include securement structures 470.

Securement structures 470 may interact or contact tissue at a target site and may grip onto tissue at the target site to secure distal portion 408 of guidewire 402 to the target site. Securement structures 470 may particularly assist with retaining distal portion 408 within the target site as an introduction device (e.g., an endoscope) is removed. Securement structures 470, along with distal portion 408 of guidewire 402, may be released from a target site once a sufficient force (e.g., proximal force) is applied.

FIG. 5 shows an adaptor 505 for use with any of the aspects described above. In examples, tube 122 may only include main portion 143, including open-ended lumens 134 and 136 defined by portions of outer wall 138 and portions of inner wall 140, and may exclude branches 144 and 146 discussed above. Adaptor 505 may be configured to be removably coupled to a proximal end of such a tube 122 and may include components having similar functions as branches 144 and 146. Adaptor 505 may include a first tube or main tube 507 internally divided along its entire length, such that main tube 507 may have two separate lumens extending therethrough. Main tube 507 of adaptor 505 may be coupled to two separate tubes 515, 517 at a proximal end of main tube 507 via a first connector 513 of adaptor 505. For example, a first lumen of main tube 507 of adaptor 505 may be coupled to and in fluid communication with a lumen of a second tube 515 of adaptor 505. A second lumen of main tube 507 of adaptor 505 may be coupled to and in fluid communication with a lumen of a third tube 517.

Adaptor 505 may further include a second connector 509 coupled to a distal end of main tube 507. Second connector 509 may be configured to couple to the proximal end of main portion 143, such that the lumens of adaptor 505 may be in fluid communication with first lumen 134 and second lumen 136 of main portion 143. For example, the lumen of second tube 515 and the first lumen of main tube 507 may be in fluid communication with first lumen 134 of main portion 143 to receive guidewire 102. The lumen of third tube 517 and the second lumen of main tube 507 may be in fluid communication with second lumen 136 of main portion 143 to supply negative pressure to a foam, such as foam 106, at a distal end of main portion 143. For example, second tube 515 may have a similar function as first branch 144 and third tube 517 may have a similar function as second branch 146. In examples, third tube 517 may be configured to be coupled to a source of suction.

It will be apparent to those skilled in the art that various modifications and variations may be made in the disclosed systems, devices, and methods without departing from the scope of the disclosure. Other aspects of the disclosure will be apparent to those skilled in the art from consideration of the specification and practice of the features disclosed herein. It is intended that the specification and examples be considered as exemplary only.

We claim:

1. A medical system comprising:
a guidewire including a distal portion and a proximal portion, wherein the distal portion is configured to transition from an expanded configuration to a straightened configuration, and wherein, in the expanded configuration, the distal portion is configured to anchor to a target site in an anatomy; and
a porous body coupled to a distal end of a tube, wherein the tube includes a first lumen and a second lumen, wherein the first lumen is configured to receive at least the proximal portion of the guidewire, and wherein the second lumen is configured to be coupled to a source of suction.

2. The medical system of claim 1, wherein, in the expanded configuration, the distal portion has a sinuous, coil, or spiral shape.

3. The medical system of claim 1, wherein the distal portion includes a plurality of securement structures.

4. The medical system of claim 3, wherein the plurality of securement structures include at least one of a burr, a barb, a ridge, a protrusion, or an adhesive.

5. The medical system of claim 1, wherein the proximal portion of the guidewire includes a portion that is straight.

6. The medical system of claim 1, wherein the distal portion is configured to transition from the expanded configuration to the straightened configuration when a proximal end of the distal portion moves proximally relative to a distal end of the distal portion.

7. The medical system of claim 1, wherein the proximal portion and the distal portion are integrally formed.

8. The medical system of claim 1, wherein, in the expanded configuration, the distal portion is wider in a direction perpendicular to a longitudinal axis of the proximal portion than the proximal portion is.

9. The medical system of claim 1, wherein the distal portion is biased to the expanded configuration.

10. The medical system of claim 1, wherein the tube includes a main portion including an outer wall and an inner wall, and wherein the inner wall separates the first lumen and the second lumen.

11. The medical system of claim 10, wherein a proximal end of the tube includes a first branch and a second branch extending proximally from the main portion, wherein the first lumen extends through the first branch, and wherein the second lumen extends through the second branch.

12. The medical system of claim 11, wherein the outer wall is a first outer wall, wherein the first branch includes a second outer wall and the second branch includes a third outer wall, and wherein the second outer wall of the first branch of the tube and the third outer wall of the second branch of the tube are each continuous with the first outer wall of the main portion of the tube.

13. The medical system of claim 11, wherein the second branch is configured to couple to the source of suction to deliver negative pressure to the porous body via the second lumen of the tube.

14. The medical system of claim 10, further comprising a connector configured to be removably coupled to a proximal end of the main portion, wherein the connector includes a first tube defining a third lumen and a second tube defining a fourth lumen, and wherein, in a state in which the connector is coupled to the proximal end of the main portion, the third lumen is in fluid communication with the first lumen and the fourth lumen is in fluid communication with the second lumen.

15. The medical system of claim 1, wherein a proximal end of the proximal portion of the guidewire is configured to be inserted into the porous body and into a distal opening of the first lumen.

16. A medical system comprising:
a guidewire including a distal portion and a proximal portion, wherein the distal portion is biased to an expanded configuration; and
a porous body coupled to a distal end of a tube, wherein the tube includes a first lumen and a second lumen;
wherein a proximal end of the proximal portion is configured to be inserted into a distal opening of the first lumen so that the porous body and the tube are advanced along and relative to the guidewire in order to position the porous body and the tube at a target site.

17. The medical system of claim 16, wherein, in the expanded configuration, the distal portion has a sinuous, coil, or spiral shape.

18. A method of treating a target site of a body lumen of a patient via a medical device, the method comprising:
advancing a distal portion of a guidewire into the target site, wherein the distal portion of the guidewire is biased to an expanded configuration to anchor the distal portion relative to the target site;
inserting a proximal end of the guidewire through a porous body and into an opening of a first lumen at a distal end of a tube coupled to the porous body;
moving the tube and the porous body distally along the guidewire to position the porous body proximate to the target site;
removing the guidewire from the body lumen by pulling the guidewire proximally through the porous body and through the first lumen of the tube; and
supplying a negative pressure to the porous body through a second lumen of the tube.

19. The method of claim 18, wherein removing the guidewire from the body lumen includes pushing the porous body distally while pulling the guidewire proximally.

20. The method of claim 18, wherein pulling the guidewire proximally transitions the guidewire from the expanded configuration to a straightened configuration.
